(19) United States
(12) Patent Application Publication
(10) Pub. No.: US 2016/0051155 A1
(43) Pub. Date: Feb. 25, 2016

(54) PATIENT VITAL SIGNS MONITORING SYSTEM AND VITAL SIGNS MONITOR

(71) Applicant: Kuo-Yuan CHANG, New Taipei City (TW)

(72) Inventor: Kuo-Yuan CHANG, New Taipei City (TW)

(21) Appl. No.: 14/539,518

(22) Filed: Nov. 12, 2014

(30) Foreign Application Priority Data
Aug. 19, 2014 (TW) 103214807

(51) Int. Cl. A61B 5/0205; A61B 19/00; A61B 5/00; A61B 5/0402; A61B 5/145

(52) U.S. Cl. CPC A61B 5/02055; A61B 5/0402; A61B 5/14532; A61B 5/14542; A61B 5/4878; A61B 19/44; A61B 5/0002; A61B 5/022

(57) ABSTRACT

A patient vital sign monitoring system comprises a frame, a bar code list and a vital sign monitor. The vital sign monitor uses a bar code reader to reads measurement bar codes or function-setting bar codes of the bar code list and generates an action instruction corresponding to a measurement instruction of one measurement bar code or a function-setting instruction of one function-setting bar code to a control unit of a host computer module. According to the action instruction, the control unit triggers an optional vital sign measurement unit to undertake the selected vital sign measurement and transmit the result to a database, or sets a function of the vital sign monitor. The patient vital sign monitoring system is easily transported to and flexibly applied to different wards, avoiding inaccuracy of manual transcription and errors in the transfer process, and saving the time the nursing personnel spend in transcription.

### BACKGROUND OF THE INVENTION

1. Field of the Invention

The present invention relates to a patient vital sign monitoring system, particularly to a patient vital sign monitoring system able to flexibly select the functions of a vital sign monitor according to requirements of patients or wards, and the vital sign monitors used by the system.

2. Description of the Prior Art

The medical apparatuses grow more and more advanced nowadays. In order to obtain and monitor vital signs of patients, various vital signal monitors are arranged in the clinics, emergency units, intensive care units and general wards of hospitals. At present, the vital sign monitors are mainly used to measure body temperature, blood pressure and blood oxygen. The measurement results are presented on the screens of the vital sign monitors and recorded by medical personnel if necessary. Some vital sign monitors can alert the medical personnel while the vital sign value of a patient is abnormal. If the medical personnel is not in the nearby of the patient, they may fail to treat the patient timely, however.

In addition to body temperature, blood pressure and blood oxygen, the medical personnel also have to consider measuring the following vital signs according to the condition of a patient, including pulse rate, respiration rate, electrocardiography, urine volume, stool volume, pain index, food intake, and fluid infusion. In the past, medical personnel record vital signs manually. The modern hospitals normally adopt electronic anamneses and use electronic vital sign monitors to automatically transmit vital sign data of patients. However, most of the vital signs still need to be examined, transcribed and then input into computers by medical personnel manually. Thus, medical personnel have to spend time in transcribing and inputting vital sign data with the likelihood of inaccurate manual transcription and erroneous vital sign data transference.

### SUMMARY OF THE INVENTION

One objective of the present invention is to provide a patient vital sign monitoring system, which is easily transported and flexibly applied to different wards. The vital sign monitors used by the system can execute different measurement instructions and function-setting instructions according to the requirement of the ward. The medical personnel can undertake required vital sign measurements via merely scanning the bar codes of the required measurements to the host computer module. Then, the vital sign monitor transmits the measurement results to a database. Thereby, the present invention not only can avoid the inaccuracy of manual transcription and the errors in the transfer process but also can save the time the nursing personnel spend in transcription.

To achieve the abovementioned objective, one embodiment of the present invention proposes a patient vital sign monitoring system, which comprises a frame, a bar code list, and a vital sign monitor. The bar code list is placed on one side of the frame and contains a plurality of measurement bar codes and a plurality of function-setting bar codes. The measurement bar codes respectively correspond to different measurement instructions. The function-setting bar codes respectively correspond to different function-setting instructions. The vital sign monitor is mounted on the frame. The vital sign monitor further comprises a host computer module and a bar code reader. The host computer module further comprises a control unit, at least one default vital sign measurement unit, a plurality of optional vital sign measurement units, and an intelligent memory unit. The default vital sign measurement unit is used to measure the vital signs of a patient, including a blood pressure measurement unit. The optional vital sign measurement units respectively correspond to the measurement instructions of different measurement bar codes. The optional vital sign measurement units are electrically connected with and triggered by the control unit to measure vital signs and output the measurement results. The intelligent memory unit is electrically connected with the control unit. The intelligent memory unit contains preset data and a plurality of action instructions corresponding to the measurement instructions of the measurement bar codes and the function-setting instructions of the function-setting bar codes. The bar code reader is electrically connected with the control unit, reading at least one of the measurement bar codes and the function-setting bar codes, and generating an output signal to the intelligent memory unit. After receiving the output signal, the intelligent memory unit compares the output signal with the preset data and generates an action instruction corresponding to the measurement instruction or the function-setting instruction. The intelligent memory unit further transmits the action instruction to the control unit. If the action instruction corresponds to the measurement instruction of a measurement bar code, the control unit triggers one of the optional vital sign measurement units according to the action instruction. If the action instruction corresponds to one of the function-setting instructions, the control unit sets a function of the vital sign monitor according to the action instruction. The user can use the bar code reader to read a plurality of measurement bar codes and/or a plurality of function-setting bar codes; the measurement instructions and/or the function-setting instructions can be arranged to form a menu containing a plurality of instruction combinations, whereby the user can select an instruction combination from the menu to fast undertake a desired operation.

Another embodiment of the present invention proposes a vital sign monitor, which comprises a control unit, a bar code reader, at least one default vital sign measurement unit, a plurality of optional vital sign measurement units, and an intelligent memory unit. The bar code reader is electrically connected with the control unit. The bar code reader is used to read a plurality of measurement bar codes and a plurality of function-setting bar codes. The measurement codes respectively correspond to different measurement instructions. The function-setting bar codes respectively correspond to different function-setting instructions. The default vital sign measurement unit is used to measure a vital sign of a patient, including a blood pressure measurement unit. The optional vital sign measurement units respectively correspond to the measurement instructions of different measurement bar codes. The optional vital sign measurement units are electrically connected with and triggered by the control unit to measure vital signs and output the measurement results. The intelligent memory unit is electrically connected with the control unit. The intelligent memory unit contains preset data and a plurality of action instructions corresponding to the measurement instructions of the measurement bar codes and the function-setting instructions of the function-setting bar codes. The bar code reader is electrically connected with the control unit, reading at least one of the measurement bar codes and the function-setting bar codes, and generating an output signal to the intelligent memory unit. After receiving the output signal, the intelligent memory unit compares the output signal with the preset data and generates an action instruction corresponding to the measurement instruction or the function-setting instruction. The intelligent memory unit further transmits the action instruction to the control unit. If the action instruction corresponds to the measurement instruction of a measurement bar code, the control unit triggers one of the optional vital sign measurement units according to the action instruction. If the action instruction corresponds to one of the function-setting instructions, the control unit sets a function of the vital sign monitor according to the action instruction. The user can use the bar code reader to read a plurality of measurement bar codes and/or a plurality of function-setting bar codes; the measurement instructions and/or the function-setting instructions can be arranged to form a menu containing a plurality of instruction combinations, whereby the user can select an instruction combination from the menu to fast undertake a desired operation.

### BRIEF DESCRIPTION OF THE DRAWINGS

The foregoing aspects and many of the accompanying advantages of this invention will become more readily appreciated as the same becomes better understood by reference to the following detailed descriptions, when taken in conjunction with the accompanying drawings, wherein:

FIG. 1 is a diagram schematically showing the structure of a patient vital sign monitoring system according to one embodiment of the present invention;

FIG. 2 is a block diagram schematically showing a host computer of a vital sign monitor according to one embodiment of the present invention; and

FIG. 3 is a diagram schematically showing the application of a patient vital sign monitoring system according to one embodiment of the present invention.

### DESCRIPTION OF THE PREFERRED EMBODIMENT

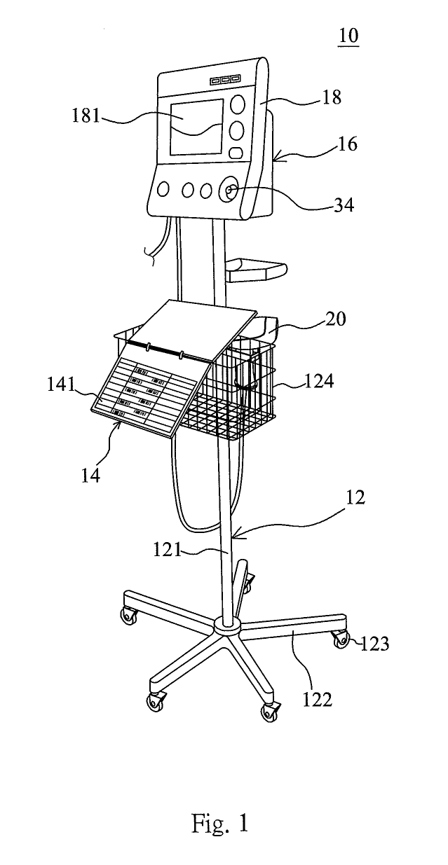

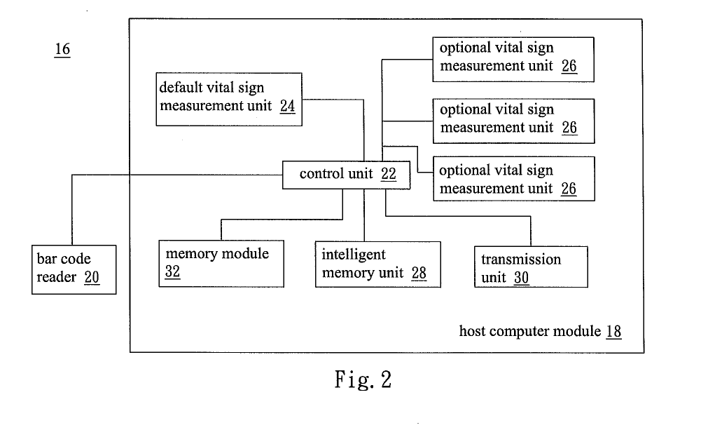

Refer to FIG. 1 a diagram schematically showing the structure of a patient vital sign monitoring system according to one embodiment of the present invention. The patient vital sign monitoring system 10, of the present invention comprises a frame 12, a bar code list 14, and a vital sign monitor 16. The bar code list 14 is placed on one side of the frame 12 and contains a plurality of measurement bar codes and a plurality of function-setting bar codes. The measurement bar codes respectively correspond to different measurement instructions. The function-setting bar codes respectively correspond to different function-setting instructions. The vital sign monitor 16 further comprises a host computer module 18 mounted on the frame 12 and a bar code reader 20 electrically connected with the host computer module 18. Refer to FIG. 2 a block diagram schematically showing a host computer module of a vital sign monitor according to one embodiment of the present invention. The host computer module 18 further comprises a control unit 22, at least one default vital sign measurement unit 24, a plurality of optional vital sign measurement units 26, and an intelligent memory unit 28. The default vital sign measurement unit 24 is electrically connected with the control unit 22 and used to measure a vital sign of a patient, including a blood pressure measurement unit that cooperates with a cuff to measure the blood pressure of a patient. The optional vital sign measurement units 26 respectively correspond to the measurement instructions of different measurement bar codes on the bar code list 14, used to measure different vital signs and output the measurement results. The optional vital sign measurement units 26 are electrically connected with and triggered by the control unit 22 to measure vital signs and output the measurement results. The intelligent memory unit 28 is electrically connected with the control unit 22. The intelligent memory unit 28 contains preset data and a plurality of action instructions. The action instructions correspond to the measurement instructions of the measurement bar codes and the function-setting instructions of the function-setting bar codes.

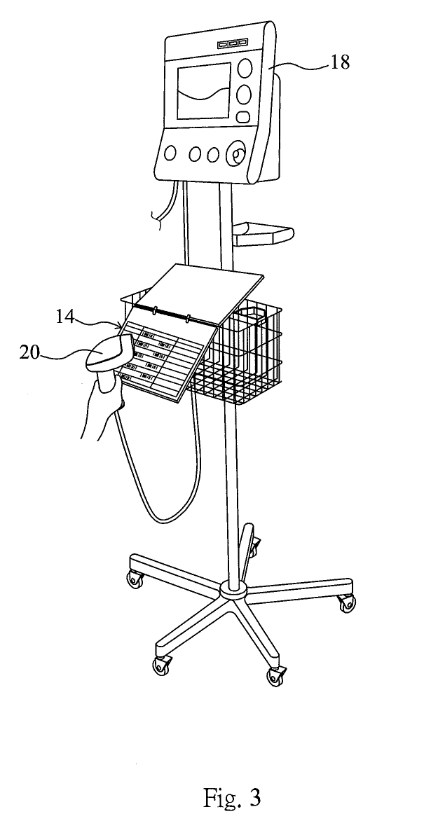

Refer to FIG. 1 and FIG. 2 again, and refer to FIG. 3. The bar code reader 20 is electrically connected with the control unit 22, reading at least one of the measurement bar codes and the function-setting bar codes of the bar code list 14, and generating an output signal to the intelligent memory unit 28. After receiving the output signal, the intelligent memory unit 28 compares the output signal with the preset data and generates an action instruction corresponding to the measurement instruction or the function-setting instruction. After receiving the output signal, the intelligent memory unit 28 compares the output signal with the preset data and generates an action instruction corresponding to the measurement instruction or the function-setting instruction. The intelligent memory unit 28 further transmits the action instruction to the control unit 22. If the action instruction corresponds to the measurement instruction of a measurement bar code, the control unit 22 triggers one of the optional vital sign measurement units 26 according to the action instruction. If the action instruction corresponds to one of the function-setting instructions, the control unit 22 sets a function of the host computer module 18 according to the action instruction. The user can use the bar code reader 20 to read a plurality of measurement bar codes and/or a plurality of function-setting bar codes; the measurement instructions and/or the function-setting instructions can be arranged to form a menu containing a plurality of instruction combinations, whereby the user can select an instruction combination from the menu to fast undertake a desired operation. In one embodiment, the host computer module 18 of the vital sign monitor 16 further comprises a transmission unit 30 electrically connected with the control unit 22.

Refer to FIG. 1 again. In one embodiment, the frame 12 further comprises a longitudinal rod 121, a fixing seat 122 connected with the bottom of the longitudinal rod 121, wheels 123 arranged on the legs of the fixing seat 122, and a rack 124 arranged on an appropriate position of the middle section of the longitudinal rod 121. On the rack 124 may be placed the bar code reader 20 and measurement accessories (not shown in the drawings), such as the cuff for blood pressure measurement, the thermometer of temperature measurement, the lancets for blood glucose measurement, the sensor for electrocardiography measurement, and the sensor for blood oxygen measurement. In one embodiment, the bar code list 14 includes a plurality of tags 141 where a plurality of measurement bar codes and a plurality of function-setting bar codes are printed; the bar code list 14 is hanged on the rack 124. In one embodiment, the measurement bar codes and the function-setting bar codes are arranged on one side of the casing of the host computer module 18.

Table. 1 shows a plurality of measurement bar codes according to one embodiment of the present invention. The measurement bar codes respectively correspond to different measurement instructions, such as the instructions of measuring urinary protein, urinary sugar, urinary ketone, blood glucose, blood oxygen, edema, body weight, body height, body temperature, stool volume, and pain indexes. While the bar code reader 20 reads one of the measurement bar codes, such as the bar code of measuring blood glucose, the measurement instruction corresponding to the measure bar code is presented on a display 181 of the host computer module 18 and triggers one optional vital sign measurement unit 26 of the vital sign monitor 16 (shown in FIG. 2) corresponding to the measurement instruction, such as the blood glucose measurement unit. Thus, the medical personnel can use the blood glucose measurement unit and a lancet (not shown in the drawings) to measure the blood glucose of a patient. The vital sign monitor 16 uses the transmission unit 30 (shown in FIG. 2) to automatically transmit the measured vital signal value to a database. Alternatively, the medical personnel inputs the measured vital sign value into the vital sign monitor 16, and the system transmits the measured vital sign value to a database through the transmission unit 30.

TABLE 1

| Test Item | Bar Code | Content |
|---|---|---|
| PR03-RPRO | [symbol] | Urinary Protein |
| PR04-UGLU | [symbol] | Urinary Sugar |
| PR05-EDEMA | [symbol] | Edema |
| PR06-WEIGHT | [symbol] | Body Weight |
| PR07-UKET | [symbol] | Urinary Ketone |
| PR08-BGLU | [symbol] | Blood Glucose |
| PR09-HEIGHT | [symbol] | Height |
| PR10-PAIN | [symbol] | Pain Index |
| PR11-STOOL | [symbol] | Stool Volume |

In one embodiment, the default vital sign measurement unit 24 is a blood oxygen measurement unit, an electrocardiography measurement unit, or a body temperature measurement unit, which measures the blood oxygen, the electrocardiography or the body temperature of a patient with a blood oxygen sensor, an electrocardiography sensor, or a body temperature sensor. The present invention does not particularly limit that the default vital sign measurement unit 24 or the optional vital sign measurement units 26 must include what type of vital sign measurement unit. In some embodiments, the abovementioned blood oxygen measurement unit, electrocardiography measurement unit, or body temperature measurement unit may be alternatively used as the optional vital sign measurement unit 26; the measurement bar codes corresponding to blood oxygen measurement, electrocardiography measurement and body temperature are printed on the bar code list 14. While one of the abovementioned optional measurements is required, the user can use the bar code reader 20 to read the corresponding bar code to the host computer module 18. In the present invention, the measured vital sign information, such the information of blood pressure, blood oxygen or electrocardiography, can be processed by the Fast Fourier Transform (FFT) to obtain the pulse spectrum.

The different function-setting bar codes respectively correspond to different function-setting instructions, such as the function-setting instructions of resetting, volume control, wireless transmission, wireless link, manual input, pressure control, measurement for children, screen brightness control, software versions, engineering modes, patient data input, user authorization, calibration, plotting, and database. While the bar code reader reads the function-setting bar code of resetting for executing the instruction of resetting, the control unit turns off the optional vital sign measurement units that are turned on originally, whereby only the default vital sign measurement units are kept ready for work. While the bar code reader reads the function-setting bar code of volume control, the volume of the speaker can be decreased or increased gradually, or the speaker is silenced completely. While the bar code reader reads the function-setting bar codes of wireless transmission and wireless link, the vital sign monitor 16 can function as a wireless access point, whereby the user can wirelessly link a smart phone to the vital sign monitor 16 and uses the smart phone to undertake function setting. While the bar code reader reads the function-setting bar code of manual input, the user can inputs more types of vital signs; for example, the user can additionally input the respiration rate.

The present invention uses the transmission unit 30 to transmit the vital sign information and/or at least one measurement result of the optional vital sign measurement units 26 to a database, whereby the present invention not only can avoid the inaccuracy of manual transcription and the errors in the transfer process but also can save the time the nursing personnel spend in transcription. In one embodiment, the database is a cloud database, a Hospital Information System (HIS), or a Nursing Information System (NIS); the database or information system can automatically check whether the vital sign information is a critical value according to a standard; the vital sign information or the critical value is automatically reported to the physician or the nursing station in a wired or wireless method, such as email, Short Message Service (SMS), or Multimedia Messaging Service. As shown in FIG. 2, the host computer module 18 further includes a memory module 32 electrically connected with the control unit 22 and used to store the vital sign information and/or at least one measurement result of the optional vital sign measurement units 26. The memory module 32 includes an external storage device, which is a memory chip or a memory card. Besides, a portable electronic device, which is electrically connected with the vital sign monitor 16, can be used to store the vital sign information and/or at least one measurement result of the optional vital sign measurement units 26.

In the present invention, the user can manually input the measurement instructions and the function-setting instructions via using a turning knob 34 on the host computer module 18, in addition to via using the bar code reader 20. The host computer module 18 also has LED alert lights for providing alert signals.

In the present invention, the vital sign monitor 16 and the bar code list 14 are placed on the mobile frame 12. Therefore, the patient vital sign monitoring system 10 can be easily moved to and flexibly applied to different wards. The vital sign monitor 16 can execute different measurement instructions and different function-setting instructions according to the requirements of different wards. The medical personnel only need to scan the corresponding bar code to the host computer module 18 before they intend to undertake a measurement or input vital sign values manually. Then, the measurement results or the vital signal values are transmitted to a database. For example, pain index measurement is necessary for the patients in cancer wards, but it is not necessary for the patients in general wards. The patient vital sign monitoring system 10 for cancer wards may use the vital sign monitor 16 to undertake measurement and surveillance of pain indexes. The patient vital sign monitoring system 10 for general wards needn't undertake pain index measurement. Thereby, the vital sign monitor 16 is exempted from having many measurement functions set therein, which frees the personnel from the inconvenience of selecting in many functions and favors their operation.

The abovementioned embodiments have been used to demonstrate the technical thought and characteristics of the present invention to enable the persons skilled in the art to understand, make, and use the present invention. However, these embodiments are only to exemplify the present invention but not to limit the scope of the present invention. Any equivalent modification or variation according to the spirit of the present invention is to be also included within the scope of the present invention.

What is claimed is:

1. A patient vital sign monitoring system comprising
a frame;
a bar code list placed on one side of said frame and including a plurality of measurement bar codes and a plurality of function-setting bar codes, wherein said measurement bar codes respectively correspond to different measurement instructions, and said function-setting bar codes respectively correspond to different function-setting instructions; and
a vital sign monitor mounted on said frame and including a host computer module and a bar code reader,
wherein said host computer module further includes
a control unit;
at least one default vital sign measurement unit, including a blood pressure measurement unit, electrically connected with said control unit, and used to measure vital signs of a patient;
a plurality of optional vital sign measurement units respectively corresponding to said measurement instructions of said measurement bar codes for measuring or inputting different vital signs, electrically connected with said control unit, and triggered by said control unit; and
an intelligent memory unit electrically connected with said control unit, and having preset data and a plurality of action instructions respectively corresponding to said measurement instructions of said measurement bar codes and said function-setting instructions of said function-setting bar codes; and
wherein said bar code reader is electrically connected with said control unit, reads at least one of said measurement bar codes or said function-setting bar codes, and generates an output signal to said intelligent memory unit, wherein after receiving said output signal, said intelligent memory unit compares said output signal with said preset data and generates one said action instruction corresponding to one said measurement instruction or one said function-setting instruction; said intelligent memory unit transmits said action instruction to said control unit; if said action instruction corresponds to one of said measurement instructions of said measurement bar codes, said control unit triggers one of said optional vital sign measurement units according to said action instruction; if said action instruction corresponds to one of said function-setting instructions of said function-setting bar codes, said control unit sets a function of said vital sign monitor according to said action instruction.

2. The patient vital sign monitoring system according to claim 1, wherein said vital sign monitor further includes a transmission unit electrically connected with said control unit and transmitting vital sign information and/or at least one measurement result of said optional vital sign measurement units to a database.

3. The patient vital sign monitoring system according to claim 1, wherein said measurement instructions include instructions of measuring urinary protein, urinary sugar, urinary ketone, blood glucose, blood oxygen, edema, body weight, body height, body temperature, stool volume, and pain indexes.

4. The patient vital sign monitoring system according to claim 1, wherein said default vital sign measurement unit is a blood oxygen measurement unit, an electrocardiography measurement unit, or a body temperature measurement unit.

5. The patient vital sign monitoring system according to claim 1, wherein said function-setting instructions include a resetting instruction; while executing said resetting instruction, said control unit turns off said optional vital sign measurement units.

6. The patient vital sign monitoring system according to claim 1, wherein said function-setting instructions include instructions of volume control, pressure control, measurement for children, screen brightness control, software versions, engineering modes, medical personnel data input, patient data input, user authorization, calibration, plotting, and database.

7. The patient vital sign monitoring system according to claim 1, wherein said function-setting instructions include instructions of wireless transmission and wireless link, which enable said vital sign monitor to function as a wireless access point.

8. The patient vital sign monitoring system according to claim 1 further comprising a portable electronic device electrically connected with said vital signal monitor and storing said vital sign information and/or at least one measurement result of said optional vital sign measurement units.

9. The patient vital sign monitoring system according to claim 1, wherein said vital sign monitor further comprises a memory module electrically connected with said control unit and used to store said vital sign information and/or at least one measurement result of said optional vital sign measurement units, and wherein said memory module includes an external storage medium, which is a memory chip or a memory card.

10. The patient vital sign monitoring system according to claim 1, wherein said vital sign monitor further comprises a turning knob that medical personnel use to manually input said measurement instructions or said function-setting instructions.

11. A vital sign monitor comprising
a control unit;
a bar code reader electrically connected with said control unit, reading at least one of a plurality of measurement bar codes and a plurality of function-setting bar codes, wherein said measurement bar codes respectively correspond to different measurement instructions, and said function-setting bar codes respectively correspond to different function-setting instructions;
at lest one default vital sign measurement unit, including a blood pressure measurement unit, electrically connected with said control unit, and used to measurement vital signs of a patient;
a plurality of optional vital sign measurement units respectively corresponding to said measurement instructions of said measurement bar codes for measuring or inputting different vital signs, electrically connected with said control unit, and triggered by said control unit; and
an intelligent memory unit electrically connected with said control unit, and having preset data and a plurality of action instructions respectively corresponding to said measurement instructions of said measurement bar codes and said function-setting instructions of said function-setting bar codes,
wherein after reading at least one of said measurement bar codes or said function-setting bar codes, said bar code reader generates an output signal to said intelligent memory unit; after receiving said output signal, said intelligent memory unit compares said output signal with said preset data and generates one said action instruction corresponding to one said measurement instruction or one said function-setting instruction; said intelligent memory unit transmits said action instruction to said control unit; if said action instruction corresponds to one of said measurement instructions of said measurement bar codes, said control unit triggers one of said optional vital sign measurement units according to said action instruction; if said action instruction corresponds to one of said function-setting instructions of said function-setting bar codes, said control unit sets a function of said vital sign monitor according to said action instruction.

12. The vital sign monitor according to claim 11 further comprising a transmission unit electrically connected with said control unit and transmitting vital sign information and/or at least one measurement result of said optional vital sign measurement units to a database.

13. The vital sign monitor according to claim 11, wherein said measurement instructions include instructions of measuring urinary protein, urinary sugar, urinary ketone, blood glucose, blood oxygen, edema, body weight, body height, body temperature, stool volume, and pain indexes.

14. The vital sign monitor according to claim 11, wherein said default vital sign measurement unit is a blood oxygen measurement unit, an electrocardiography measurement unit, or a body temperature measurement unit.

15. The vital sign monitor according to claim 11, wherein said function-setting instructions include a resetting instruction; while executing said resetting instruction, said control unit turns off said optional vital sign measurement units.

16. The vital sign monitor according to claim 11, wherein said function-setting instructions include instructions of volume control, pressure control, measurement for children, screen brightness control, software versions, engineering modes, medical personnel data input, patient data input, user authorization, calibration, plotting, and database.

17. The vital sign monitor according to claim 11, wherein said function-setting instructions include instructions of wireless transmission and wireless link, which enable said vital sign monitor to function as a wireless access point.

18. The vital sign monitor according to claim 11 further comprising a memory module electrically connected with said control unit and used to store said vital sign information and/or at least one measurement result of said optional vital sign measurement units, and wherein said memory module includes an external storage medium, which is a memory chip or a memory card.

19. The vital sign monitor according to claim 11 further comprising a turning knob that medical personnel use to manually input said measurement instructions or said function-setting instructions.
